(19) United States
(12) Patent Application Publication
(10) Pub. No.: US 2022/0370468 A1
(43) Pub. Date: Nov. 24, 2022

(54) Methods of treating sleep disorders associated with pain

(71) Applicant: Astellas Pharma Global Development, Inc., Northbrook, IL (US)

(72) Inventors: Paul Blahunka, Lake Bluff, IL (US); Gerard Marek, Vernon Hills, IL (US); Katherine Tracy, Sonoma, CA (US); Mary Beth Blauwet, Lindenhurst, IL (US); Michael Smith, Ann Arbor, MI (US); Mark Walzer, River Forest, IL (US); Marci English, Clarendon Hills, IL (US)

(21) Appl. No.: 17/627,993

(22) PCT Filed: Jul. 22, 2020
(86) PCT No.: PCT/US2020/043047

§ 371 Date: Jan. 18, 2022

Related U.S. Application Data
(60) Provisional application No. 62/877,573, filed on Jul. 23, 2019.

(51) Int. Cl. A61K 31/5377; A61P 25/20

(52) U.S. Cl. CPC A61K 31/5377; A61P 25/20

(57) ABSTRACT

Provided herein in some embodiments are methods of treating a sleep disorder associated with pain in a patient in need thereof, comprising administering to the patient a compound of Formula (I), or a pharmaceutically acceptable salt thereof. Provided herein in some embodiments are methods of treating a sleep disorder associated with pain in a patient in need thereof, comprising: a) identifying a patient having a sleep disorder associated with pain; and b) administering to the patient identified in step a) a compound of Formula (I), or a pharmaceutically acceptable salt thereof. Provided herein in some embodiments are methods of treating a sleep disorder associated with pain in a patient in need thereof, comprising: a) selecting a patient having a sleep disorder associated with pain; and b) administering to the patient identified in step a) a compound of Formula (I), or a pharmaceutically acceptable salt thereof. In some embodiments, the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night. In some embodiments, the sleep disorder is associated with pain selected from the group consisting of fibromyalgia, neuropathic pain, osteoarthritis, and visceral pain. In some more particular embodiments, the visceral pain is selected from the group consisting of IBS associated pain and bladder pain.

### CROSS REFERENCE TO RELATED APPLICATIONS

The present application claims priority to U.S. Provisional Application No. 62/877,573, filed on Jul. 23, 2019, the disclosure of which is incorporated by reference herein in its entirety.

### FIELD OF THE INVENTION

The present disclosure relates to methods for treating sleep disorders associated with pain. In particular, the present disclosure in some embodiments relates to methods for treating sleep disorders associated with pain that comprise administering the compound of Formula (I), or a pharmaceutically acceptable salt thereof, to a patient in need thereof.

### BACKGROUND

The dramatic increase in deaths from opioid-induced overdoses (fentanyl and other opioids) has set in motion a number of responses to this crisis, including the NIH Helping to End Addiction Over the Long-term (HEAL) initiative (Volkow and Collins, 2017; Collins et al., 2018). The two overarching components of the HEAL initiative are to improve treatments for opioid use disorders (including overdose-reversal interventions) and to enhance pain management. While only a minority of patients with chronic pain become addicted to opioids, prescription opioids for pain management puts patients at increased risk for addiction, overdose, and death (NIH, 2017). Thus, development of novel non-opioid treatments for chronic pain syndromes is one crucial direction in combating the ongoing opioid crisis.

Fibromyalgia occurs in approximately 2.2% of the general population in the United States (Queiroz, 2013). It is characterized by chronic widespread pain, which often occurs with fatigue and sleep disturbances (Wolfe et al., 1990; Wolfe et al., 2010a; Wolfe et al., 2011). The US Food and Drug Administration (FDA) has approved three medications for the treatment of fibromyalgia: pregabalin, duloxetine, and milnacipran. Pregabalin is an alpha-2-delta calcium channel ligand; duloxetine and milnacipran are serotonin and norepinephrine reuptake inhibitors (Arnold et al., 2012). Despite the approval of these medications, a number of opioid analgesics continue to be used, despite the lack of evidence of their effectiveness for fibromyalgia.

Given the ongoing opioid crisis in the United States, non-opioid treatments for the treatment of disorders or conditions associated with fibromyalgia and other types of pain would be desirable.

### SUMMARY

Provided herein in some embodiments are methods of treating a sleep disorder associated with pain in a patient in need thereof, comprising administering to the patient a compound of Formula (I), or a pharmaceutically acceptable salt thereof.

Provided herein in some embodiments are methods of treating a sleep disorder associated with pain in a patient in need thereof, comprising:
a) Identifying and/or selecting a patient having a sleep disorder associated with pain; and
b) administering to the patient identified in step a) a compound of Formula (I), or a pharmaceutically acceptable salt thereof.

Provided herein in some embodiments are methods of treating a patient having a sleep disorder associated with pain, comprising administering to the patient a compound of Formula (I), or a pharmaceutically acceptable salt thereof.

Provided herein in some embodiments are methods of treating a patient, comprising:
a) identifying and/or selecting a patient having a sleep disorder associated with pain; and
b) administering to the patient identified in step a) a compound of Formula (I), or a pharmaceutically acceptable salt thereof.

In some embodiments the compound of Formula (I), or a pharmaceutically acceptable salt thereof, is comprised in a pharmaceutical composition which further comprises a pharmaceutically acceptable carrier. Thus, in some embodiments of the methods herein, in the administering step the patient is administered a composition comprising a compound of Formula (I), or a pharmaceutically acceptable salt thereof, and a pharmaceutically acceptable carrier.

### BRIEF DESCRIPTION OF THE DRAWINGS

FIG. 1 shows the change from baseline in mean daily average pain score during double-blind treatment period and follow-up period (full analysis set).

FIG. 2 shows the change from baseline during double-blind treatment period and follow-up period in FIQR total (full analysis set).

FIG. 3A shows the change from baseline during double-blind treatment period and follow-up period in FIQR subscales (full analysis set)—Symptom subscale.

FIG. 3B. Change from baseline during double-blind treatment period and follow-up period in FIQR subscales (full analysis set)—Overall Impact subscale.

FIG. 3C shows the change from baseline during double-blind treatment period and follow-up period in FIQR subscales (full analysis set)—Function subscale.

FIG. 4A shows the change from baseline during double-blind treatment period and follow-up in mean daily average FMSD Item 1: Difficulty with falling asleep (full analysis set).

FIG. 4B shows the change from baseline during double-blind treatment period and follow-up in mean daily average FMSD Item 2: Restlessness of sleep (full analysis set).

FIG. 5 shows an MMRM analysis of Change from Baseline Overtime in FMSD Item 1 (Difficulty with falling asleep).

FIG. 6 shows an MMRM analysis of Change from Baseline Overtime in FMSD Item 2 (Restlessness of sleep).

FIG. 7 shows an MMRM analysis of Change from Baseline Overtime in FMSD Item 3 (Difficulty getting comfortable).

FIG. 8 shows an MMRM analysis of Change from Baseline Overtime in FMSD Item 4 (Difficulty staying asleep).

FIG. 9 shows an MMRM analysis of Change from Baseline Overtime in FMSD Item 5 (Degree of deep sleep).

FIG. 10 shows an MMRM analysis of Change from Baseline Overtime in FMSD Item 6 (Degree of being rested when waking up for the day).

FIG. 11 shows an MMRM analysis of Change from Baseline Overtime in FMSD Item 7 (Difficulty with beginning the day).

FIG. 12 shows an MMRM analysis of Change from Baseline Overtime in FMSD Item 8 (Degree of having enough sleep during the previous night).

### DETAILED DESCRIPTION

### Definitions

The “compound of Formula (I)” is the compound having the formula:

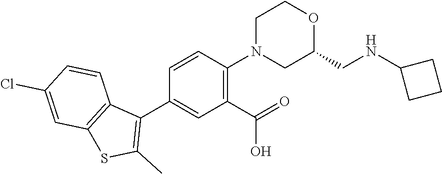 [SMILES: Cc1sc2cc(Cl)ccc2c1-c1ccc(N2CCO[C@H](CNC3CCC3)C2)c(C(=O)O)c1]

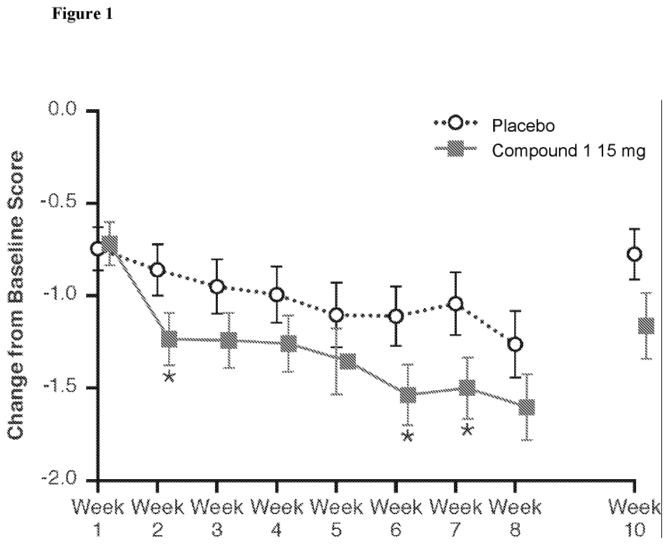

The recitation “Compound of Formula (I)” is used interchangeably with the recitation “Compound 1” in the specification and in FIGS. 1-12 herein.

The compound of Formula (I) is a non-opioid agent and a Kca3.1 potassium ion channel opener that reverses abnormal nerve firing. Physiologically, Kca3.1 is thought to regulate cellular excitability, thus making Kca3.1 channels a potential therapeutic target in diseases associated with abnormal nerve excitation. The compound of Formula (I) demonstrates poor brain penetration by design and had both an acceptable toxicological profile, as well as acceptable absorption, distribution, metabolism, and excretion (ADME) profile in nonclinical studies.

The compound of Formula (I) is disclosed in, for example, U.S. Pat. Nos. 8,981,119 and 9,399,038, the contents of each of which are incorporated by reference herein in their entirety.

In some embodiments described herein, the pharmaceutically acceptable salt of the compound of Formula I is the hydrobromide salt, shown below:

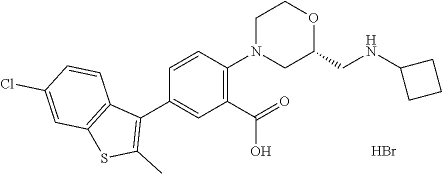 [SMILES: Cc1sc2cc(Cl)ccc2c1-c1ccc(N2CCO[C@H](CNC3CCC3)C2)c(C(=O)O)c1 ; Br]

The term “administration” or “administering” refers to a method of providing a dosage of a compound or pharmaceutical composition comprising the compound to a patient. In some embodiments, the patient is a human. In some embodiments, the patient is a non-human mammal. The compositions according to the present invention may be administered orally in solid dosage forms, such as capsules, tablets, and powders, or in liquid dosage forms, such as elixirs, syrups and suspensions. Furthermore the compositions containing the therapeutic agents may be administered parenterally, in sterile liquid dosage forms, by transmucosal delivery via solid, liquid or aerosol forms or transdermally via a patch mechanism, cream, lotion or ointment. Various types of transmucosal administration include respiratory tract mucosal administration, nasal mucosal administration, oral transmucosal (such as sublingual and buccal) administration, and rectal transmucosal administration.

The term “treating” (or “treat” or “treatment”) refers to alleviating, abating or ameliorating a disorder or condition or symptoms thereof, preventing additional symptoms, ameliorating the underlying causes of symptoms, inhibiting the disorder or condition, e.g., arresting the development of the disorder or condition, relieving the disorder or condition, causing regression of the disorder or condition, relieving a condition caused by the disorder or condition, or stopping the symptoms of the disorder or condition therapeutically. For example, the term “treating” in reference to a disorder may include a reduction in severity of one or more symptoms associated with a particular disorder.

In some implementations, the term “treating” as used herein refers to improving or ameliorating an undesired symptom of a sleep disorder or the incidence of the sleep disorder. In some implementations, the treatment reduces the severity of the symptom of the sleep disorder by at least 15%, at least 20%, at least 25%, at least 30%, at least 35%, at least 40%, or at least 50%. In some implementations, the treatment reduces the incidence of the sleep disorder by at least 15%, at least 20%, at least 25%, at least 30%, at least 35%, at least 40%, or at least 50%. In some implementations, the reduction of severity of a symptom of a sleep disorder is measured by subjective patient feedback. In some implementations, the reduction of severity of a symptom of a sleep disorder is measured by objective measurements. In some implementations, the reduction of severity of a symptom of a sleep disorder is measured by determining the change in the value of a clinical score following administration of the compound of formula (I).

As used herein, “patient” refers to any subject, particularly a mammalian subject, for whom diagnosis, prognosis, or therapy is desired, for example, a human. In some embodiments, the patient is a patient being treated for pain. In some more particular embodiments, the patient is a patient being treated for pain with an opioid. In some embodiments, the patient is a patient being treated for pain who experiences a sleep disorder. In some more particular embodiments, the patient is a patient being treated for pain with an opioid who experiences a sleep disorder.

The terms “treatment regimen” and “dosing regimen” are used interchangeably to refer to the dose and timing of administration of the compound of formula (I).

As used herein, identifying and/or selecting a patient having a sleep disorder associated with pain” refers to identifying the patient; selecting the patient; or both identifying and selecting a patient. In some embodiments of the methods of treating a sleep disorder associated with pain, the methods comprise identifying the patient. In some embodiments, the methods comprise selecting the patient. In some embodiments, the methods comprise identifying and selecting the patient.

Reference to a sleep disorder or disorders “associated with pain” means a sleep disorder or disorders associated with pain or a pain disorder. Examples of pain disorders are fibromyalgia, neuropathic pain, osteoarthritis, and visceral pain, such as IBS associated pain and bladder pain.

Reference to “about” a value or parameter herein includes embodiments that are directed to that value or parameter. For example, “about X” includes the disclosure of “X.” Unless otherwise specified, the term “about” refers to the indicated value of the variable and to all values of the variable that are within the experimental error of the indicated value or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

Provided herein in some embodiments are methods of treating a sleep disorder associated with pain in a patient in need thereof, comprising administering to the patient a compound of Formula (I), or a pharmaceutically acceptable salt thereof.

Provided herein in some embodiments are methods of treating a sleep disorder associated with pain in a patient in need thereof, comprising:
a) identifying a patient having a sleep disorder associated with pain; and
b) administering to the patient identified in step a) a compound of Formula (I), or a pharmaceutically acceptable salt thereof.

Provided herein in some embodiments are methods of treating a sleep disorder associated with pain in a patient in need thereof, comprising:
a) selecting a patient having a sleep disorder associated with pain; and
b) administering to the patient identified in step a) a compound of Formula (I), or a pharmaceutically acceptable salt thereof.

Provided herein in some embodiments are methods of treating a patient having a sleep disorder associated with pain, comprising administering to the patient a compound of Formula (I), or a pharmaceutically acceptable salt thereof.

Provided herein in some embodiments are methods of treating a patient, comprising:
a) identifying a patient having a sleep disorder associated with pain; and
b) administering to the patient identified in step a) a compound of Formula (I), or a pharmaceutically acceptable salt thereof.

Provided herein in some embodiments are methods of treating a patient, comprising:
a) selecting a patient having a sleep disorder associated with pain; and
b) administering to the patient identified in step a) a compound of Formula (I), or a pharmaceutically acceptable salt thereof.

In some more particular embodiments, methods, as provided herein, comprise administering a compound of Formula (I), or a pharmaceutically acceptable salt thereof, to a patient twice a day, daily, every other day, three times a week, twice a week, weekly, every other week, twice a month, or monthly. In some embodiments, the method comprises administering the compound of Formula I, or a pharmaceutically acceptable salt thereof, to a patient daily.

In some embodiments, the amount administered is titrated from a lower dose to a higher dose over a period of time. In some embodiments, the amount administered is titrated from a higher dose to a lower dose over a period of time.

In some more particular embodiments, a method as provided herein comprises administering a compound of Formula (I), or a pharmaceutically acceptable salt thereof, to a patient over a period of time. The period of time may be, for example, based on one or more of: the stage of disease in the patient, the mass and sex of the patient, age of the patient, clinical trial guidelines, and information on the approved drug label if applicable. In some embodiments, a suitable period of time can be from 1 week to 2 years, such as 1 week, 2 weeks, 3 weeks, 4 weeks, 6 weeks, 8 weeks, 12 weeks, 3 months, 24 weeks, 6 months, 12 months, 18 months, or 2 years, or any value in between. In other embodiments, a suitable period of time can be from 1 month to 10 years, for example, 1 month, 6 months, 1 year, 2 years, 3 years, 4 years, 5 years, 6 years, 7 years, 8 years, 9 years, or 10 years, or any value in between.

In some embodiments, the compound of Formula (I) is administered in a therapeutically effective amount. The term “therapeutically effective amount” with reference to a compound disclosed herein is an amount that is sufficient to achieve the desired therapeutic effect. In some embodiments, the compound of Formula (I) is administered in a therapeutically effective amount to improve sleep or one or more symptoms of a sleep disorder.

In some embodiments, the compound of Formula (I) is administered in an amount equal to about 5 mg to about 45 mg per day, such as about 5 mg per day, about 10 mg per day, about 15 mg per day, about 20 mg per day, about 25 mg per day, about 30 mg per day, about 35 mg per day, about 40 mg per day, or about 45 mg per day.

In some embodiments, the compound of Formula (I) is administered in an amount that is effective to treat the sleep disorder associated with pain, wherein the amount is not effective to treat the pain.

As used herein, an amount of compound of Formula (I) is not effective to treat pain where that amount does not provide a reduction from baseline in pain numerical rating scale (“NRS”) of more than 30%, more than 40%, or more than 50%. Alternatively, the amount of the compound of Formula (1) can be administered in an amount that reduces the pain NRS by no more than 1.9 points, such as no more than 1.8 points, such as no more than 1.7 points, such as no more than 1.6 points, such as no more than 1.5 points.

The amount of the compound of Formula (I) may be administered in one or more doses, such as the total amount per day is the desired amount. For example, an amount of 15 mg per day of the compound of Formula (I) may be administered in one dose of 15 mg per day, or in two doses each of 7.5 mg per day, or in three doses each of 5 mg per day. The dosage, or the dosage per day, may differ between the different clinical visits or visits to a medical practitioner based on an observation or measurement of the severity of the disorder or condition being treated.

In some embodiments, the compound of Formula (I) is administered in a dose equal to about 5 mg to about 30 mg, such as about 5 mg, about 10 mg, about 15 mg, about 20 mg, about 25 mg, or about 30 mg.

In some embodiments, the compound of Formula (I) is administered in a dose that is effective to treat the sleep disorder associated with pain, wherein the dose is not effective to treat the pain

In some embodiments of the methods disclosed herein, the sleep disorder is measured using a sleep diary, wrist actigraphy, or polysomnography.

In some embodiments, the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night.

In some embodiments, the sleep disorder is measured using a sleep diary, and the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night.

In some embodiments, the sleep disorder is measured using wrist actigraphy or polysomnography, and the sleep disorder selected from the group consisting of difficulty with falling asleep; restlessness of sleep; difficulty staying asleep; degree of deep sleep.

In some embodiments, the sleep disorder is difficulty with falling asleep.

In some embodiments, the sleep disorder is restlessness of sleep.

In some embodiments of the methods disclosed herein, the method reduces the severity of a symptom of the sleep disorder.

In some embodiments, the sleep disorder is associated with pain selected from the group consisting of fibromyalgia, neuropathic pain, osteoarthritis, and visceral pain. In some more particular embodiments, the visceral pain is selected from the group consisting of IBS associated pain and bladder pain.

Accordingly, provided herein in some embodiments are methods of treating a sleep disorder associated with pain, wherein the pain is selected from the group consisting of fibromyalgia, neuropathic pain, osteoarthritis, and visceral pain, such as IBS associated pain and bladder pain, in a patient in need thereof, comprising administering to the patient a compound of Formula (I), or a pharmaceutically acceptable salt thereof.

Provided herein in some embodiments are methods of treating a sleep disorder associated with pain, wherein the pain is selected from the group consisting of fibromyalgia, neuropathic pain, osteoarthritis, and visceral pain, such as IBS associated pain and bladder pain, in a patient in need thereof, comprising:
a) identifying a patient having a sleep disorder associated with the pain selected from the group consisting of fibromyalgia, neuropathic pain, osteoarthritis, and visceral pain, such as IBS associated pain and bladder pain; and
b) administering to the patient identified in step a) a compound of Formula (I), or a pharmaceutically acceptable salt thereof.

Provided herein in some embodiments are methods of treating a sleep disorder associated with pain, wherein the pain is selected from the group consisting of fibromyalgia, neuropathic pain, osteoarthritis, and visceral pain, such as IBS associated pain and bladder pain, in a patient in need thereof, comprising:
a) selecting a patient having a sleep disorder associated with the pain selected from the group consisting of fibromyalgia, neuropathic pain, osteoarthritis, and visceral pain, such as IBS associated pain and bladder pain; and
b) administering to the patient identified in step a) a compound of Formula (I), or a pharmaceutically acceptable salt thereof.

Provided herein in some embodiments are methods of treating a patient having a sleep disorder associated with pain, wherein the pain is selected from the group consisting of fibromyalgia, neuropathic pain, osteoarthritis, and visceral pain such as IBS associated pain and bladder pain, comprising administering to the patient a compound of Formula (I), or a pharmaceutically acceptable salt thereof.

Provided herein in some embodiments are methods of treating a patient, comprising:
a) identifying a patient having a sleep disorder associated with the pain selected from the group consisting of fibromyalgia, neuropathic pain, osteoarthritis, and visceral pain, such as IBS associated pain and bladder pain; and
b) administering to the patient identified in step a) a compound of Formula (I), or a pharmaceutically acceptable salt thereof.

Provided herein in some embodiments are methods of treating a patient, comprising:
a) selecting a patient having a sleep disorder associated with the pain selected from the group consisting of fibromyalgia, neuropathic pain, osteoarthritis, and visceral pain, such as IBS associated pain and bladder pain; and
b) administering to the patient identified in step a) a compound of Formula (I), or a pharmaceutically acceptable salt thereof.

Provided herein in some embodiments are methods of treating a sleep disorder associated with fibromyalgia in a patient in need thereof, comprising administering to the patient a compound of Formula (I), or a pharmaceutically acceptable salt thereof.

Provided herein in some embodiments are methods of treating a sleep disorder associated with fibromyalgia in a patient in need thereof, comprising:
a) identifying a patient having a sleep disorder associated with fibromyalgia; and
b) administering to the patient identified in step a) a compound of Formula (I), or a pharmaceutically acceptable salt thereof.

Provided herein in some embodiments are methods of treating a sleep disorder associated with fibromyalgia in a patient in need thereof, comprising:
a) selecting a patient having a sleep disorder associated with fibromyalgia; and
b) administering to the patient identified in step a) a compound of Formula (I), or a pharmaceutically acceptable salt thereof.

Provided herein in some embodiments are methods of treating a patient having a sleep disorder associated with fibromyalgia, comprising administering to the patient a compound of Formula (I), or a pharmaceutically acceptable salt thereof.

Provided herein in some embodiments are methods of treating a patient, comprising:
a) identifying a patient having a sleep disorder associated with fibromyalgia; and
b) administering to the patient identified in step a) a compound of Formula (I), or a pharmaceutically acceptable salt thereof.

Provided herein in some embodiments are methods of treating a patient, comprising:
a) selecting a patient having a sleep disorder associated with fibromyalgia; and
b) administering to the patient identified in step a) a compound of Formula (I), or a pharmaceutically acceptable salt thereof.

In some embodiments of the methods disclosed herein, the patient is a patient who has been previously administered a sleeping aid. In some embodiments, the sleeping aid can be a non-benzodiazopene sedative hypnotic (e.g., zolpidem, zaleplon, eszopliclone and the like), a benzodiazapene like sleep aid (such as temazepam, triazolam, lorazepam, flunazepam, quazepam, clonazepam, and the like), a melatonin like sleep aid (such as ramelteon, melatonin and the like), a sedating tricyclic antidepressant (such as amitriptyline, doxepine, nortriptyline, imipramine, and the like), low doses of antipsychotic drugs such as quetiapine, clozapine and the like, antidepressants used as sleep aids (such as trazodone, mirtzapine, and the like), dual orexin receptor antagonists (such as suvorexant and the like), and/or a sedating histamine-1 receptor antagonist (such as diphenhydramine and the like).

In some embodiments of the methods disclosed herein, the patient is a patient for whom treatment with a sleeping aid such as a non-benzodiazopene sedative hypnotic (such as zolpidem, zaleplon, eszopliclone and the like), a benzodiazapene like sleep aid (such as temazepam, triazolam, lorazepam, flunazepam, quazepam, clonazepam, and the like), a melatonin like sleep aid (such as ramelteon, melatonin and the like), a sedating tricyclic antidepressant (such as amitriptyline, doxepine, nortriptyline, imipramine, and the like), low doses of antipsychotic drugs such as quetiapine, clozapine and the like, antidepressants used as sleep aids (such as trazodone, mirtzapine, and the like), dual orexin receptor antagonists (such as suvorexant and the like), and/or a sedating histamine-1 receptor antagonist (such as diphenhydramine and the like) is contraindicated.

In some embodiments of the methods herein, the pain in the patient is a pain that is being treated with one or more of

(a) a pain reliever selected from the group consisting of gabapentinoids (including pregabalin, gabapentin), antidepressants (except for serotonin reuptake inhibitors, including serotonin reuptake inhibitors selected from duloxetine, venlafaxine, milnacipran, tricyclic antidepressants (including tricyclic antidepressants selected from amitriptyline and nortriptyline), trazodone, nefazodone, mirtazapine, and bupropion), ketamine, esketamine and other NMDA receptor blocking drugs, GABAB receptor agonists (including sodium oxybate and baclofen), opioids (including morphine, fentanyl, codeine, hydrocodone, oxycodone, hydromorphone, and tramadol), celecoxib and meloxicam, muscle relaxants such as cyclobenzaprine, cannabis and cannabinoids (including cannabinoids containing THC), NSAIDS (including acetaminophen or acetaminophen-containing formulations, ibuprofen, and naproxen), chronic non-narcotic analgesics (other than aspirin for cardioprophylaxis in a dose up to 325 mg daily), and topical pain medications (including capsaicin)

or

(b) a procedure for pain relief selected from the group consisting of electrical stimulation including spinal cord stimulation or transcutaneous electrical nerve stimulation), acupuncture, nerve block, iontophoresis, laser therapy, tender point injections, dry needle injections, chiropractic treatment, exercise or physical therapy, surgical therapy, and biofeedback.

In some embodiments of the methods herein, the patient is being treated with one or more of

(a) a pain reliever selected from the group consisting of gabapentinoids (including pregabalin, gabapentin), antidepressants (except for serotonin reuptake inhibitors, including serotonin reuptake inhibitors selected from duloxetine, venlafaxine, milnacipran, tricyclic antidepressants (including tricyclic antidepressants selected from amitriptyline and nortriptyline), trazodone, nefazodone, mirtazapine, and bupropion), ketamine, esketamine and other NMDA receptor blocking drugs, GABAB receptor agonists (including sodium oxybate and baclofen), opioids (including morphine, fentanyl, codeine, hydrocodone, oxycodone, hydromorphone, and tramadol), celecoxib and meloxicam, muscle relaxants such as cyclobenzaprine, cannabis and cannabinoids (including cannabinoids containing THC), NSAIDS (including acetaminophen or acetaminophen-containing formulations, ibuprofen, and naproxen), chronic non-narcotic analgesics (other than aspirin for cardioprophylaxis in a dose up to 325 mg daily), and topical pain medications (including capsaicin)

or

(b) a procedure for pain relief selected from the group consisting of electrical stimulation including spinal cord stimulation or transcutaneous electrical nerve stimulation), acupuncture, nerve block, iontophoresis, laser therapy, tender point injections, dry needle injections, chiropractic treatment, exercise or physical therapy, surgical therapy, and biofeedback.

Also provided herein in some embodiments is a compound of Formula (I), or a pharmaceutically acceptable salt thereof, for use in a method for treating a sleep disorder associated with pain.

Provided herein in some embodiments is a compound of Formula (I), or a pharmaceutically acceptable salt thereof, for use in a method for treating a sleep disorder associated with pain in a patient that has been identified as having a sleep disorder associated with pain.

Provided herein in some embodiments is the use of a compound of Formula (I), or a pharmaceutically acceptable salt thereof, in the manufacture of a medicament for treating a sleep disorder associated with pain.

Provided herein in some embodiments is the use of a compound of Formula (I), or a pharmaceutically acceptable salt thereof, in the manufacture of a medicament for treating a sleep disorder associated with pain in a patient that has been identified as having a sleep disorder associated with pain.

Also provided herein in some embodiments is a compound of Formula (I), or a pharmaceutically acceptable salt thereof, for use in a method for treating a sleep disorder associated with pain, wherein the pain is selected from the group consisting of fibromyalgia, neuropathic pain, osteoarthritis, and visceral pain, such as IBS associated pain and bladder pain.

Provided herein in some embodiments is a compound of Formula (I), or a pharmaceutically acceptable salt thereof, for use in a method for treating a sleep disorder associated with pain, wherein the pain is selected from the group consisting of fibromyalgia, neuropathic pain, osteoarthritis, and visceral pain, such as IBS associated pain and bladder pain, in a patient that has been identified as having a sleep disorder associated with the pain.

Provided herein in some embodiments is the use of a compound of Formula (I), or a pharmaceutically acceptable salt thereof, in the manufacture of a medicament for treating a sleep disorder associated with pain, wherein the pain is selected from the group consisting of fibromyalgia, neuropathic pain, osteoarthritis, and visceral pain, such as IBS associated pain and bladder pain.

Provided herein in some embodiments is the use of a compound of Formula (I), or a pharmaceutically acceptable salt thereof, in the manufacture of a medicament for treating a sleep disorder associated with pain, wherein the pain is selected from the group consisting of fibromyalgia, neuropathic pain, osteoarthritis, and visceral pain, such as IBS associated pain and bladder pain, in a patient that has been identified as having a sleep disorder associated with the pain.

Also provided herein in some embodiments is a compound of Formula (I), or a pharmaceutically acceptable salt thereof, for use in a method for treating a sleep disorder associated with pain, wherein the sleep disorder is measured using a sleep diary, wrist actigraphy, or polysomnography.

Provided herein in some embodiments is a compound of Formula (I), or a pharmaceutically acceptable salt thereof, for use in a method for treating a sleep disorder associated with pain, wherein the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night.

Provided herein in some embodiments is a compound of Formula (I), or a pharmaceutically acceptable salt thereof, for use in a method for treating a sleep disorder associated with pain, wherein the sleep disorder is measured using a sleep diary, and the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night.

Provided herein in some embodiments is a compound of Formula (I), or a pharmaceutically acceptable salt thereof, for use in a method for treating a sleep disorder associated with pain, wherein the sleep disorder is measured using wrist actigraphy or polysomnography, and the sleep disorder selected from the group consisting of difficulty with falling asleep; restlessness of sleep; difficulty staying asleep; degree of deep sleep.

In some embodiments of the compound of Formula (I), or a pharmaceutically acceptable salt thereof, for use in a method for treating a sleep disorder associated with pain, the sleep disorder is difficulty with falling asleep.

In some embodiments of the compound of Formula (I), or a pharmaceutically acceptable salt thereof, for use in a method for treating a sleep disorder associated with pain, the sleep disorder is restlessness of sleep.

In some embodiments of the compound of Formula (I), or a pharmaceutically acceptable salt thereof, for use in a method for treating a sleep disorder associated with pain, the sleep disorder is associated with pain selected from the group consisting of fibromyalgia, neuropathic pain, osteoarthritis, and visceral pain. In some more particular embodiments, the visceral pain is selected from the group consisting of IBS associated pain and bladder pain.

Also provided herein in some embodiments is the use of a compound of Formula (I), or a pharmaceutically acceptable salt thereof, in the manufacture of a medicament for treating a sleep disorder associated with pain, wherein the sleep disorder is measured using a sleep diary, wrist actigraphy, or polysomnography.

Provided herein in some embodiments is the use of a compound of Formula (I), or a pharmaceutically acceptable salt thereof, in the manufacture of a medicament for treating a sleep disorder associated with pain, wherein the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night.

Provided herein in some embodiments is the use of a compound of Formula (I), or a pharmaceutically acceptable salt thereof, in the manufacture of a medicament for treating a sleep disorder associated with pain, wherein the sleep disorder is measured using a sleep diary, and the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; and degree of having enough sleep during the previous night.

Provided herein in some embodiments is the use of a compound of Formula (I), or a pharmaceutically acceptable salt thereof, in the manufacture of a medicament for treating a sleep disorder associated with pain, wherein the sleep disorder is measured using wrist actigraphy or polysomnography, and the sleep disorder selected from the group consisting of difficulty with falling asleep; restlessness of sleep; difficulty staying asleep; degree of deep sleep.

In some embodiments of the use of a compound of Formula (I), or a pharmaceutically acceptable salt thereof, in the manufacture of a medicament for treating a sleep disorder associated with pain, the sleep disorder is difficulty with falling asleep.

In some embodiments of the use of a compound of Formula (I), or a pharmaceutically acceptable salt thereof, in the manufacture of a medicament for treating a sleep disorder associated with pain, the sleep disorder is restlessness of sleep.

In some embodiments of the use of a compound of Formula (I), or a pharmaceutically acceptable salt thereof, in the manufacture of a medicament for treating a sleep disorder associated with pain, the sleep disorder is associated with pain selected from the group consisting of fibromyalgia, neuropathic pain, osteoarthritis, and visceral pain. In some more particular embodiments, the visceral pain is selected from the group consisting of IBS associated pain and bladder pain.

The severity of a sleep disorder associated with pain may be determined, for example, by measuring the value of a clinical score associated with the sleep disorder. Similarly, in some embodiments, the effectiveness of the treatment of a sleep disorder associated with pain may be determined, for example, by measuring the value of a clinical score associated with the sleep disorder following the treatment. Accordingly, in some embodiments, a method of treatment disclosed herein comprises determining the value for the patient of a clinical score associated with the sleep disorder. In some embodiments, the method comprises determining that the value of the clinical score at a time point following administration of the compound of Formula (I) is different from the value of the clinical score prior to or at the time of administration.

In some embodiments, the clinical score is a score for one or more FMSD (fibromyalgia sleep diary) items. The FMSD (Kleinman et al., 2014) is a validated 8-item patient-reported outcome that assesses sleep disturbances specific to patients with fibromyalgia across the domains of falling asleep, staying asleep, and sufficient sleep (Kleinman et al., 2014). Each day upon waking, patients rated their sleep quality over the previous night on an 11-point numerical integer rating scale (NRS). Each item is rated on an 11-point NRS, ranging from 0, which corresponds to “not at all”, to 10, which corresponds to “extremely.”

FMSD items include:
difficulty with falling asleep (improvement shown as a decrease in measurement);
restlessness of sleep (improvement shown as a decrease in measurement);
difficulty getting comfortable (improvement shown as a decrease in measurement);
difficulty staying asleep (improvement shown as a decrease in measurement);
degree of deep sleep (improvement shown as an increase in measurement);
degree of being rested when waking up for the day (improvement shown as an increase in measurement);
difficulty with beginning the day (improvement shown as a decrease in measurement); and
degree of having enough sleep during the previous night (improvement shown as an increase in measurement).

Thus, in some embodiments, a method of treatment disclosed herein comprises determining the value for the patient of one or more FMSD items. In some embodiments, the method comprises determining that the value of an FMSD item at a time point following administration of the compound of Formula (I) is different from the value of the FMSD item prior to or at the time of administration. In some embodiments, the method comprises determining that the value of an FMSD item at least 1 week, such as from 1 week to 13 weeks, following administration of the compound of Formula (I) is different from the value of the FMSD item prior to or at the time of administration.

In some embodiments, the method comprises determining that, for each of at least two FMSD items, the value of the FMSD item at a time point following administration of the compound of Formula (I) is different from the value of the FMSD item prior to or at the time of administration. In some embodiments, the method comprises determining that, for each of at least two FMSD items, the value of the FMSD item at least 1 week, such as from 1 week to 13 weeks, following administration of the compound of Formula (I) is different from the value of the FMSD item prior to or at the time of administration.

For example, in some embodiments, the method comprises determining that the value of an FMSD item at least 1 week, such as from 1 week to 13 weeks, following administration of the compound of Formula (I) improves by at least 15%, at least 20%, at least 25%, at least 30%, at least 35% or at least 40% relative to the value of the FMSD item prior to or at the time of administration.

For example, in some embodiments, the method comprises determining that for each of at least two FMSD items, the value of the FMSD item at least 1 week, such as from 1 week to 13 weeks, following administration of the compound of Formula (I) improves by at least 15%, at least 20%, at least 25%, at least 30%, at least 35% or at least 40% relative to the value of the FMSD item prior to or at the time of administration.

For example, in some embodiments, the method comprises determining that
(a) for an FMSD item selected from the group consisting of degree of deep sleep, degree of being rested when waking up for the day, and degree of having enough sleep during the previous night, the value of the FMSD item at least 1 week, such as from 1 week to 13 weeks, following administration of the compound of Formula (I) increases by at least 15%, at least 20%, at least 25%, at least 30%, at least 35% or at least 40% relative to the value of the FMSD item prior to or at the time of administration,
and
(b) for an FMSD item selected from the group consisting of difficulty with falling asleep, restlessness of sleep, difficulty getting comfortable, difficulty staying asleep, and difficulty with beginning the day, the value of the FMSD item at least 1 week, such as from 1 week to 13 weeks, following administration of the compound of Formula (I) decreases by at least 15%, at least 20%, at least 25%, at least 30%, at least 35% or at least 40% relative to the value of the FMSD item prior to or at the time of administration.

The severity of a sleep disorder associated with pain may be determined, for example, by determining the level of an objective clinical biomarker (e.g., actigraphy, polysomnography, or other sleep study).

### Example 1

A phase 2a, randomized, double-blind, placebo-controlled, parallel group study was conducted at 24 sites in the United States to assess the analgesic efficacy and safety of the compound of Formula (I) in patients with fibromyalgia. The study consisted of the following: a screening period of up to 42 days, which included a washout period and a 7-day baseline diary run-in; a 57-day double-blind randomized treatment period with site visits at Days 1, 15, 29, and 57; and a 4-week follow-up period consisting of a clinic visit after 2 weeks and a phone call after 4 weeks. The study was carried out in accordance with the Declaration of Helsinki and approved by the relevant Institutional Review Boards. All participants provided written informed consent prior to the initiation of any study procedures.

Male and female patients aged 18 to 80 years were included in the trial if they met the American College of Rheumatology (ACR) 1990 and 2010 fibromyalgia diagnostic criteria at screening (Wolfe et al., 1990; Wolfe et al., 2010b). The core diagnostic criteria for fibromyalgia are defined by the 1990 criteria (Wolfe et al., 1990), and while ACR has published newer criteria, the 1990 version is still commonly utilized in clinical trials. To meet ACR 1990 criteria, patients must have had widespread pain for at least 3 months, defined as the presence of pain on the right and left sides of the body, pain above and below the waist, and pain in the axial skeleton (cervical spine, anterior chest, thoracic spine, or low back) (Wolfe et al., 1990). They must also have had pain in at least 11 of 18 tender point sites on digital palpation performed with an approximate force of 4 kg. To meet the ACR 2010 criteria, patients needed to have a widespread pain index (WPI) score of ≥7 and a symptom severity (SS) scale score ≥5, or a WPI score 3-6 and an SS scale score ≥9 (Wolfe et al., 2010b). Symptoms must have been present at a similar level for at least 3 months and patients must have been free of any other disorder that could have explained the pain. In addition, patients must have had a pain score ≥4 on the Fibromyalgia Impact Questionnaire Revised (FIQR) pain item at screening (Bennett et al., 2009), along with a mean daily average pain score of 4-9 (inclusive) on an 11-point (0-10) numerical rating scale (NRS) during the baseline diary run-in period, and have met prespecified criteria for mean daily average pain scores.

Treatment

Eligible patients were randomized 1:1 using Interactive Response Technology to receive oral Compound of Formula (I) 15 mg (three capsules of 5 mg) or matching placebo capsules, each given once daily in the morning, with or without food, for 8 weeks.

Endpoints and Assessment

The primary efficacy endpoint for pain was change from baseline to Week 8 in mean daily average pain score assessed by the Numeric Rating Scale (NRS; 0 to 10 scale). The NRS is a generic pain assessment instrument, which consists of a single question that asks patients to record their daily average pain on an 11-point scale (0=no pain; 10=pain as bad as you can imagine) over the previous 24 hours. Patients used a handheld e-diary to report daily average pain NRS scores and to capture rescue medication use; these data were automatically transmitted to a central database. Patients completed the NRS each day from the baseline run-in period through Week 10 every evening.

Secondary efficacy endpoints included the number of patients achieving a ≥30% or ≥50% reduction in mean daily average pain score assessed by NRS (0 to 10 scale) from baseline to Week 8 and end of treatment (EOT; Day 57); change from baseline to Weeks 2, 4, 8, and EOT in the FIQR function, symptoms, and overall impact subscales; and overall improvement assessed by patient global impression of change (PGIC) (Rampakakis et al., 2015) at Weeks 2, 4, 8, and EOT.

Questionnaires on efficacy, such as FIQR and PGIC, were completed during study visits. The FIQR captures the full spectrum of problems related to fibromyalgia and the responses to therapy, ranging from activities of daily living, overall impact, and symptoms (Bennett et al., 2009). Patients answered each question on an 11-point NRS over a recall period of the last 7 days or the last time the activity was performed (for the physical function domain if the activity was not performed within the 7-day recall period). The PGIC is a self-administered 7-point Likert scale that asks patients to evaluate their fibromyalgia relative to baseline (from “very much improved” to “very much worse”) (Rampakakis et al., 2015).

Sleep disturbance was assessed as an exploratory endpoint using change from baseline to each respective week (Weeks 1-8) and from baseline to EOT in daily fibromyalgia sleep diary (FMSD) responses (Kleinman et al., 2014). The FMSD is a validated 8-item patient-reported outcome that assesses sleep disturbances specific to patients with fibromyalgia across the domains of falling asleep, staying asleep, and sufficient sleep (Kleinman et al., 2014). Each day upon waking, patients rated their sleep quality over the previous night on an 11-point NRS using their handheld e-diary. Each item is rated on an 11-point NRS anchored by “0—not at all” and “10—extremely.”

184 randomized patients (98.9%) were included in the SAF and FAS and 173 patients (93.0%) were included in the per-protocol set. The main reason for exclusion from the per-protocol set was due to current, untreated moderate or severe major depressive disorder/history of any psychotic and/or bipolar disorder (n=8). Overall, 183 patients (n=93, placebo; n=90, Compound of Formula (I)) were included in the follow-up period. The primary reasons for leaving the study prior to randomization were screening failure, patient withdrawal, and ‘other.’ In total, 156 (83.9%) patients completed planned the double-blind treatment period and 168 patients (90.3%) completed the planned follow-up period; there were no differences between treatment and placebo groups in terms of withdrawals during the double-blind treatment period.

The treatment groups were also similar with respect to fibromyalgia-related baseline characteristics. The mean of the baseline daily average pain score was 6.32 in both treatment groups, but the proportion of patients with severe baseline mean daily average pain score (≥7-10) was higher in the Compound of Formula (I) group than in the placebo group (31.1% vs 22.3%). Prior medication used to treat fibromyalgia pain and other types of pain was similar in the Compound of Formula (I) and placebo groups.

Efficacy Results

There was no statistically significant change, only a trend in favor of Compound of Formula (I), from baseline to Week 8, in mean daily average pain score (primary endpoint) for Compound of Formula (I) versus placebo (−1.60 vs −1.26; treatment difference [SE]: −0.34 [0.25]; two-sided 90% CI: −0.76, 0.07; P=0.086). Similar results were obtained in both sensitivity and per-protocol set analyses (data not shown). However, statistically significant treatment differences in favor of Compound of Formula (I) compared with placebo were observed in a change from baseline in mean daily average pain score at Weeks 2, 6, and 7 (FIG. 1).

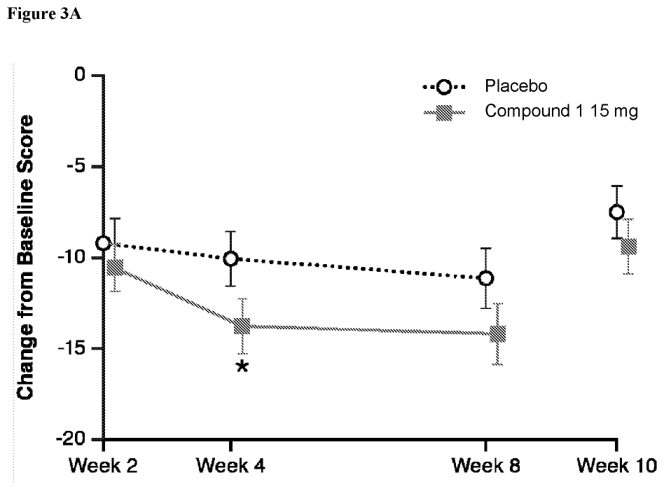

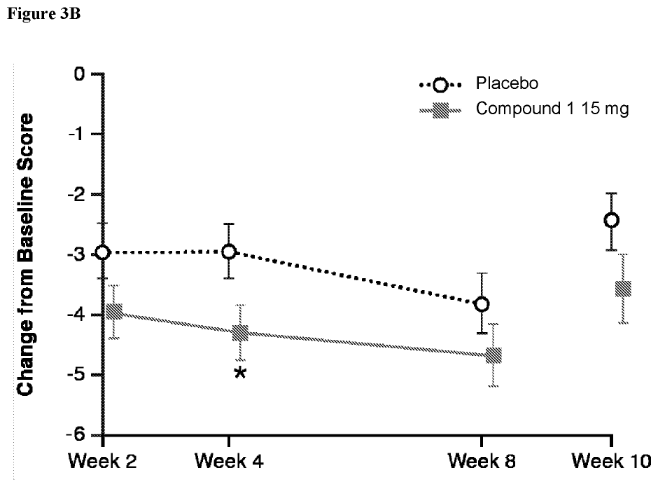

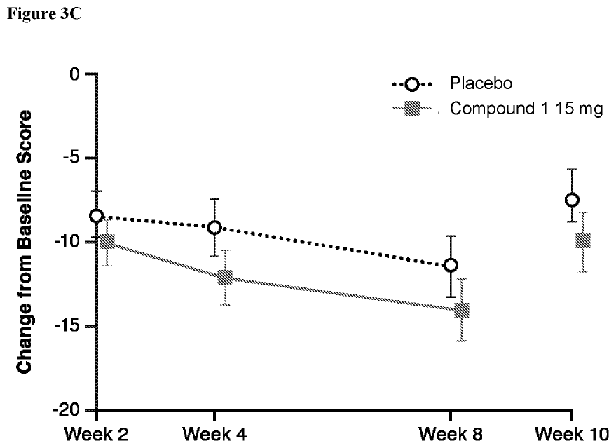

There were no statistically significant differences between Compound of Formula (I) and placebo groups in the responder analysis of mean daily average pain score or for the overall patient improvement assessed by PGIC. A statistically significant difference in favor of Compound of Formula (I) compared with placebo was seen in the symptoms subscale (FIG. 3A) and overall impact subscale (FIG. 3B) of the FIQR at Week 4 (LS mean difference: −3.73; P=0.039 and LS mean difference: −1.34; P=0.018, respectively), but not in FIQR function (LS mean difference: −2.97; P=0.103; FIG. 3C). While there was a statistically significant difference in the reduction from baseline in the total score at Week 4 (LS mean difference: −4.14; P=0.033), no statistically significant difference was seen in the reduction from baseline in FIQR total score at Week 8. There were numerical improvements for Compound of Formula (I) versus placebo, but there were no statistically significant differences in the reduction (improvement) from baseline to Week 8 in FIQR function, symptoms subscale, or overall impact subscale.

Improvement from baseline in FIQR total score was numerically higher in the Compound of Formula (I) treatment group compared with the placebo group at all time points. There was no statistically significant difference between Compound of Formula (I) and placebo in the reduction (improvement) from baseline to Week 8 in the MMRM analysis (LS mean difference: −3.27; P=0.093); however, in an ANCOVA analysis, improvement from baseline to Week 8 in FIQR total score was statistically significant for Compound of Formula (I) versus placebo using mBOCF imputation (LS mean difference: −4.10; P=0.036) and EOT using LOCF imputation (LS mean difference: −3.75; P=0.050).

In contrast with the lack of statistically significant difference observed for most of the pain scores described above, statistically significant improvements in favor of Compound of Formula (I) compared with placebo were seen in several items of the FMSD, the fibromyalgia sleep diary. Referring to FIGS. 1-4, FIG. 1 shows the mean daily average pain score assessed by the Numeric Rating Scale (NRS), which consists of a single question that asks patients to record their daily average pain on an 11-point scale (ranging from 0=no pain to 10=pain as bad as you can imagine) over the previous 24 hours. Data from double-blind period are presented as LS mean±standard error; data from follow-up period are presented as mean±standard error. The asterisk (*) in the figure indicates a value of P<0.05.

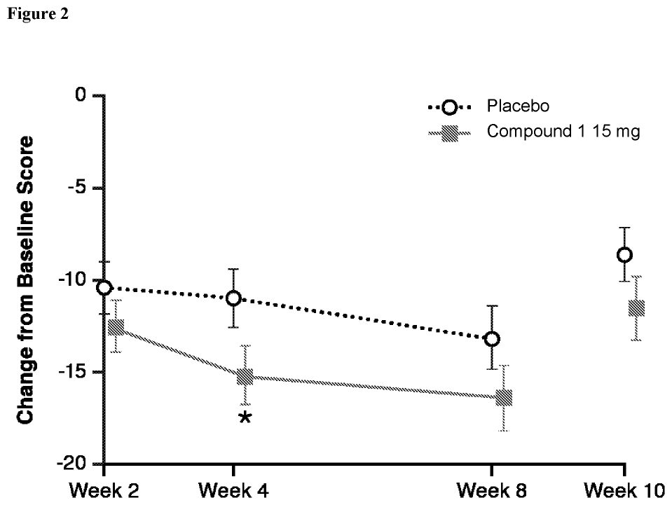

In FIG. 2, the change from baseline in FIQR total score is depicted. The FIQR total score represents the sum of the three sub scale scores: symptom, function, and overall impact. The symptom sub scale accounts for 50%, the function subscale accounts for 30%, and the overall impact subscale accounts for 20% of the total score. Data from double-blind period are presented as LS mean±standard error; data from follow-up period are presented as mean±standard error. The asterisk (*) in the figures indicates a value of P<0.05.

FIGS. 3A, 3B and 3C show the changes from baseline in the mean FIQR subscale scores: (A) Symptom, (B) Function, and (C) Overall Impact are depicted. All questions in each subscale are rated on an 11-point numeric scale, ranging from 0 to 10 with 10 being the worst. Data from double-blind period are presented as LS mean±standard error; data from follow-up period are presented as mean±standard error. The asterisk (*) in the figures indicates a value of P<0.05.

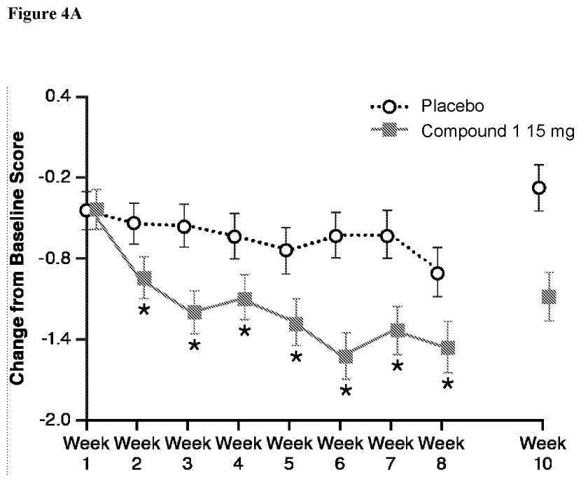

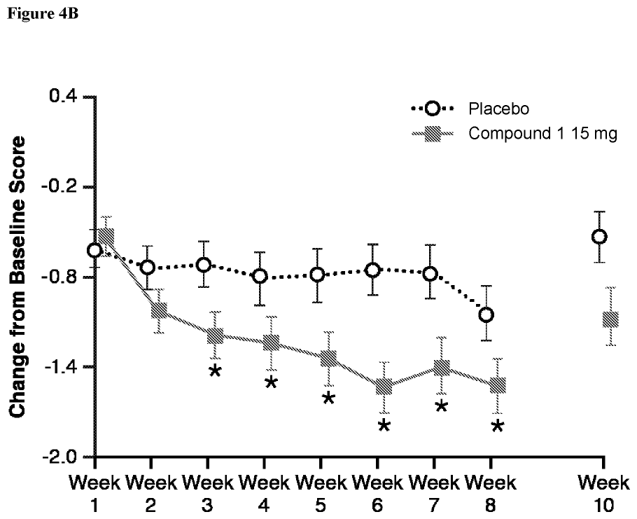

FIG. 4A shows the change from baseline in Item 1 of the FMSD, difficulty with falling asleep. FIG. 4A displays the change from baseline in Item 2 of the FMSD, restlessness of sleep. Patients rated their difficulty falling asleep or restlessness of sleep over the previous night on an 11-point numeric rating scale ranging from “0—not at all” to “10— extremely” in an e-diary. Data from double-blind period are presented as LS mean±standard error; data from follow-up period are presented as mean±standard error.

The asterisk (*) in the figures indicates a value of P<0.05.

(Referring to FIGS. 1-4, the following abbreviations are used: FIQR, Fibromyalgia Impact Questionnaire Revised; LS, least squares; FMSD, Fibromyalgia Sleep Diary.)

Statistically significant improvements were seen beginning at Week 2 (difficulty with falling asleep; P=0.032 [see FIG. 4A]), Week 3 (restlessness of sleep, P=0.013 [see FIG. 4B]; difficulty getting comfortable, P=0.007; and difficulty staying asleep, P=0.013), and Week 6 (degree of deep sleep, P=0.035; and difficulty with beginning the day, P=0.040). Statistically significant improvements for Compound of Formula (I) versus placebo were seen for difficulty with falling asleep (P=0.018) and restlessness of sleep (P=0.033) at Week 8, and for difficulty with falling asleep (P=0.008), restlessness of sleep (P=0.009), and difficulty with beginning the day (P=0.044) at EOT.

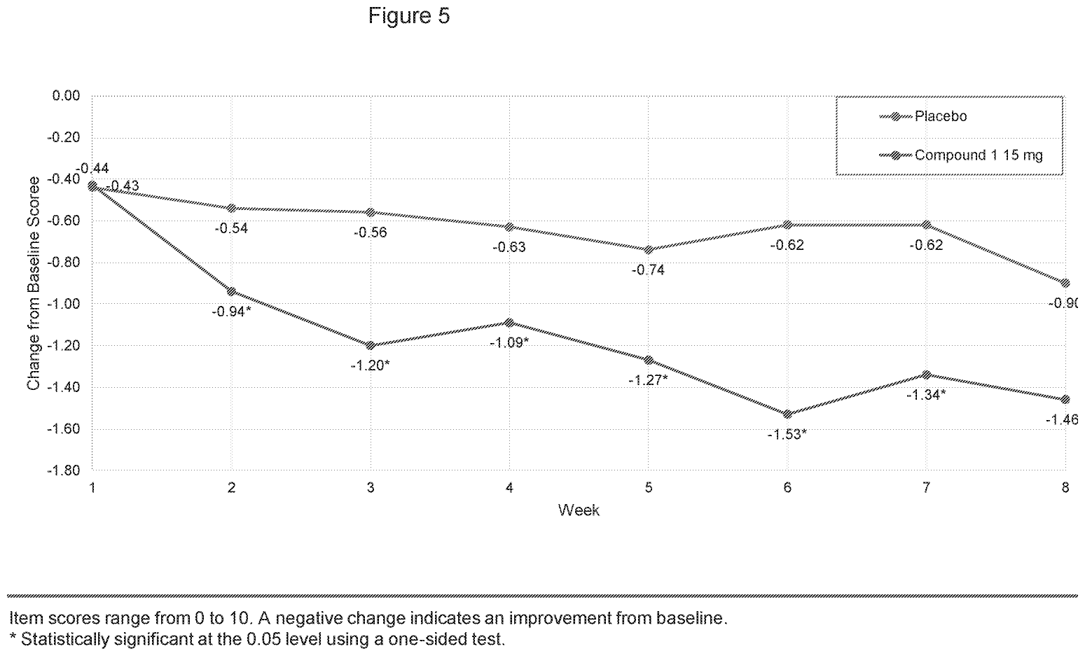

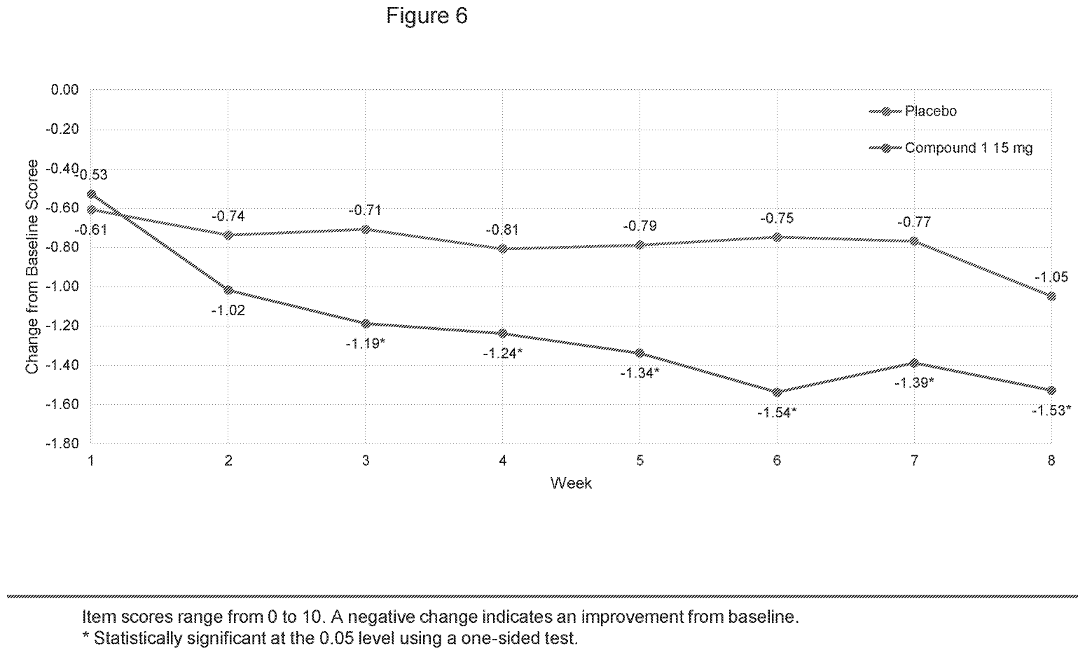

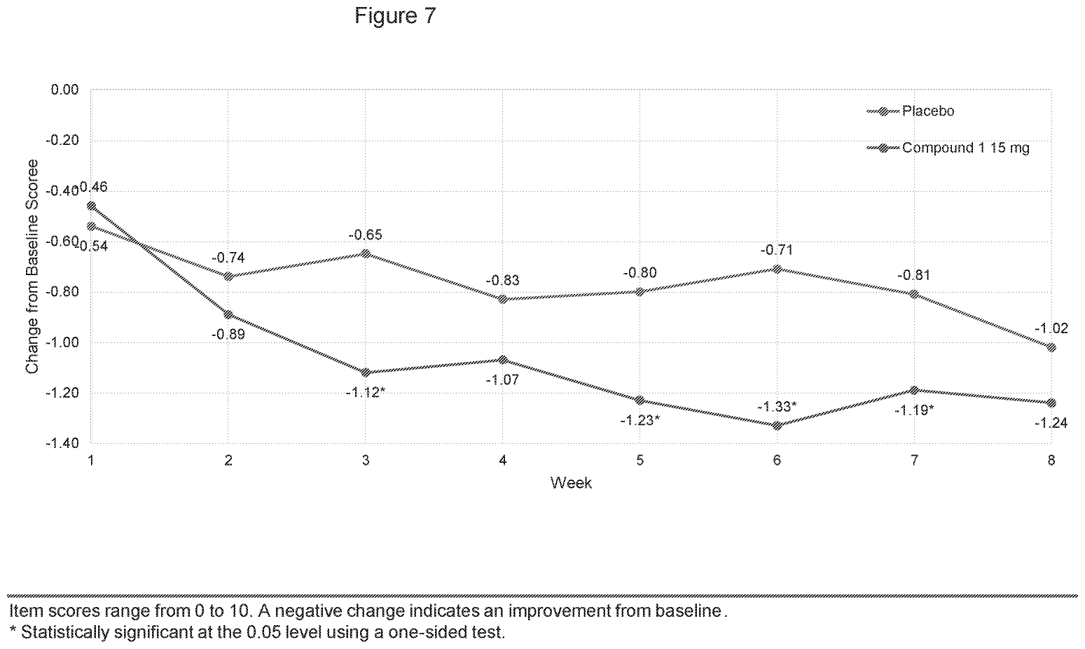

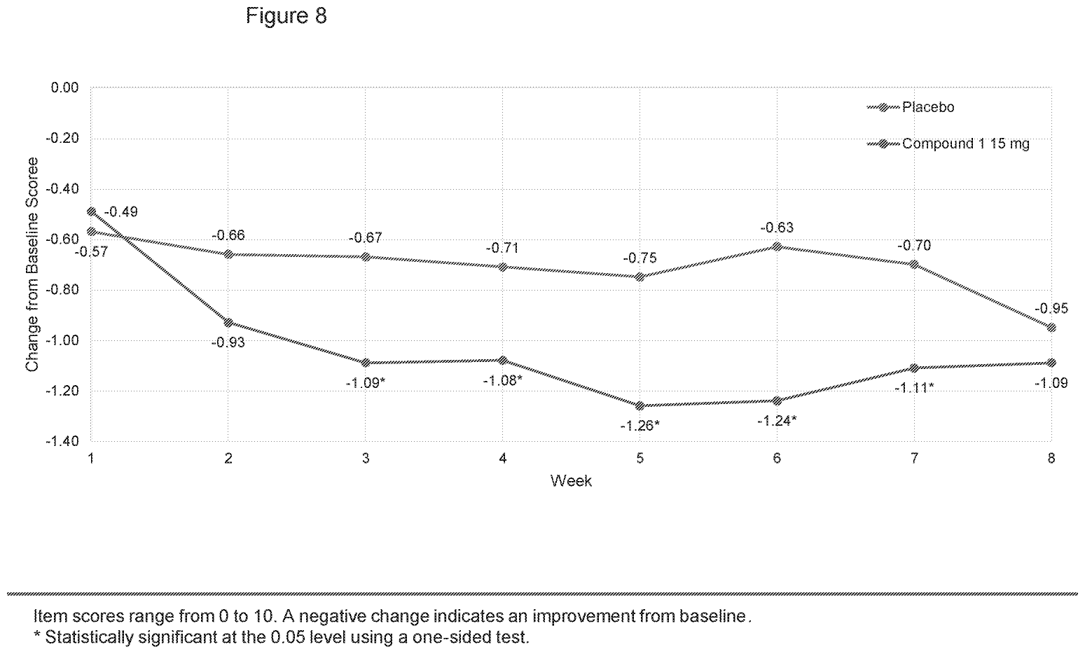

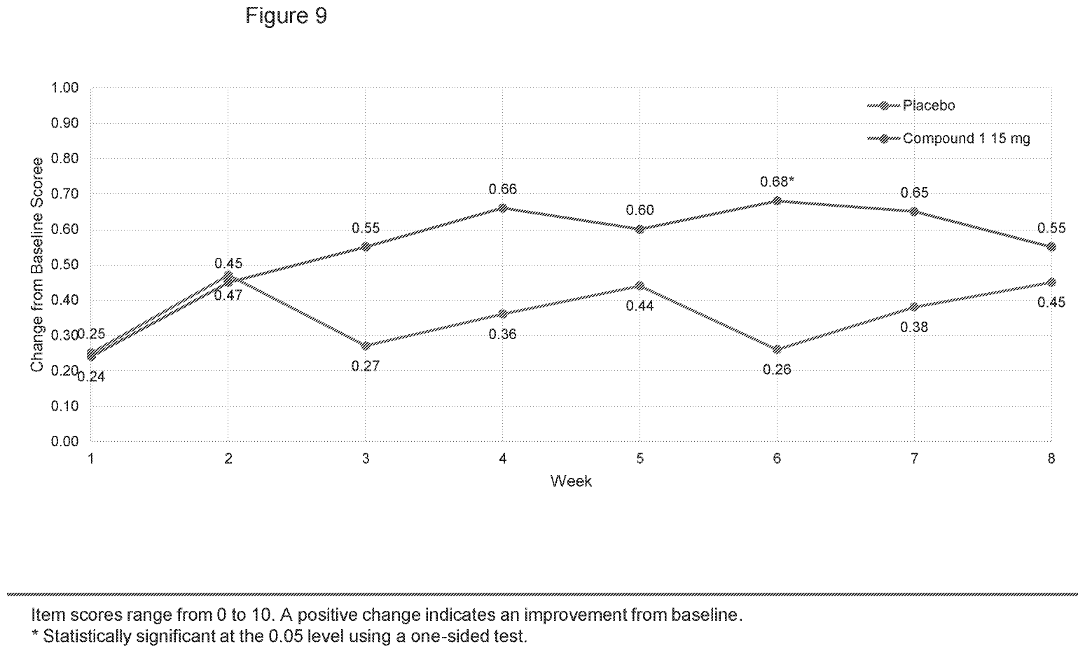

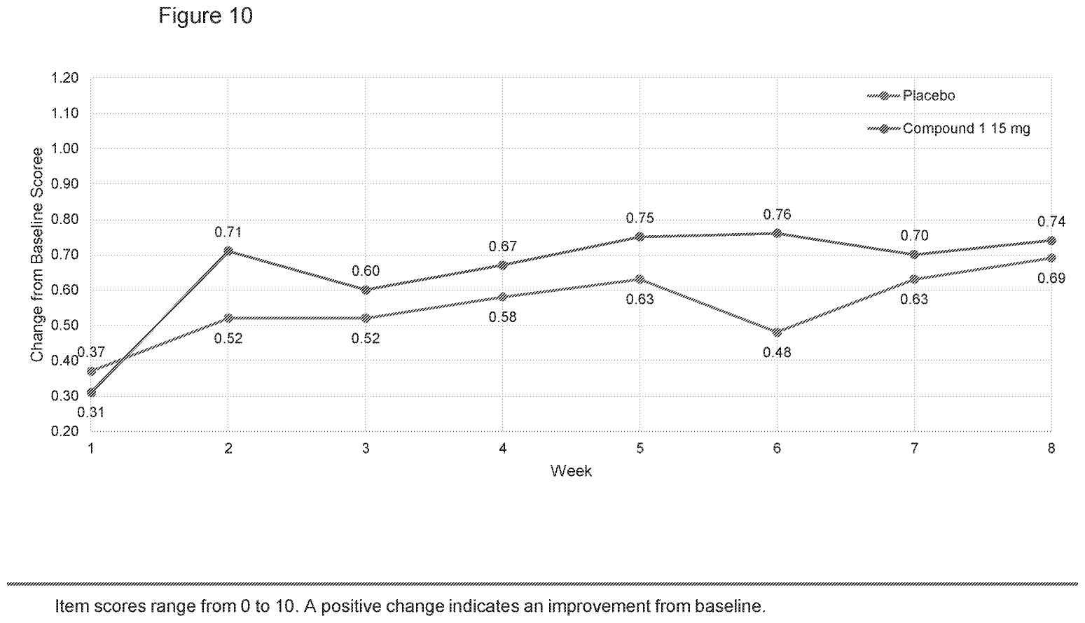

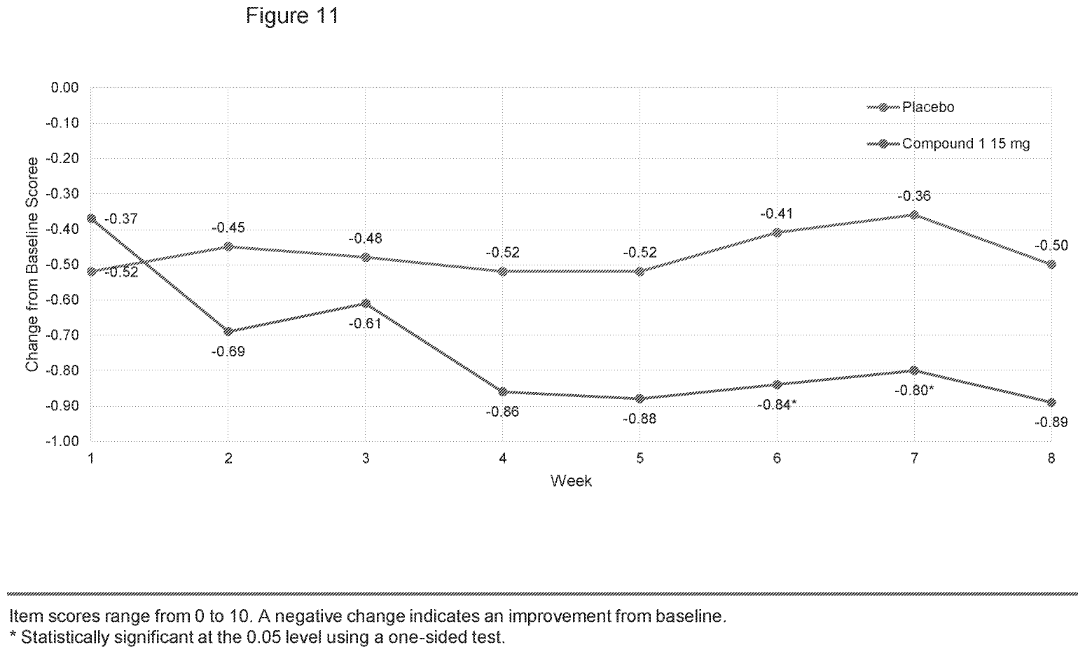

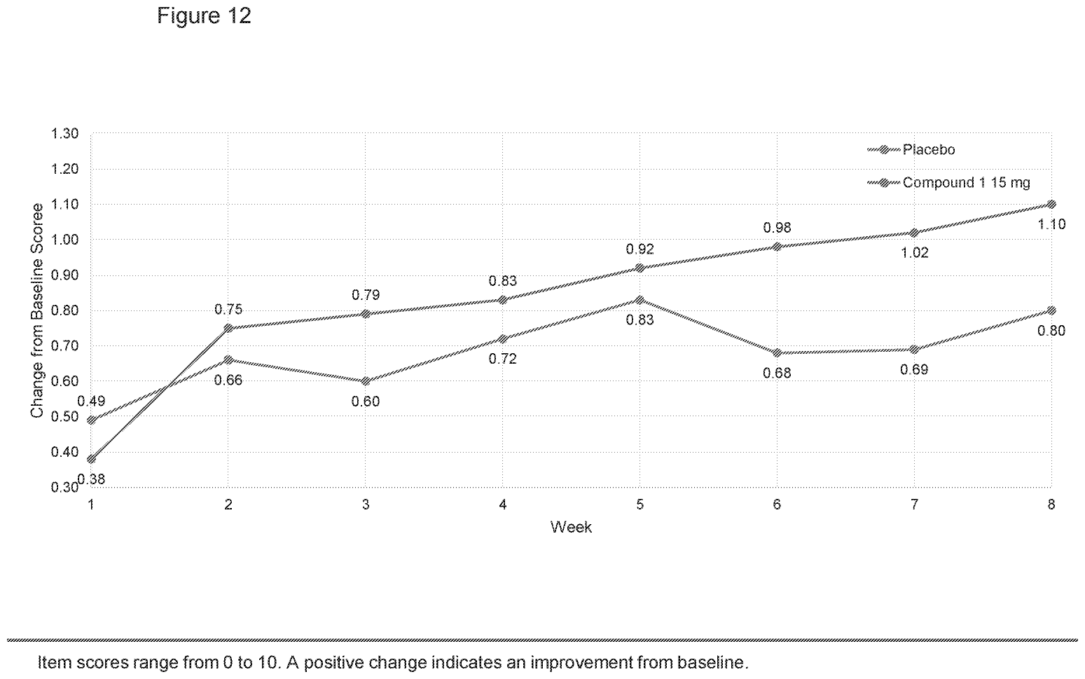

FIGS. 5-12 show additional plots of change from baseline over time for the following FMSD items:
FIG. 5: difficulty with falling asleep;
FIG. 6: restlessness of sleep;
FIG. 7: difficulty getting comfortable;
FIG. 8: difficulty staying asleep;
FIG. 9: degree of deep sleep;
FIG. 10: degree of being rested when waking up for the day;
FIG. 11: difficulty with beginning the day;
and
FIG. 12: degree of having enough sleep during the previous night.

In each of FIGS. 5-12, the respective FMSD Item scores range from 0 to 10. For FIGS. 5-8 and 11, a negative change indicates an improvement from baseline. For FIGS. 9-10 and 12, a positive change indicates an improvement from baseline. In all cases, an asterisk indicates a statistically significant value at the 0.05 level using a one-sided test.

The following tables show changes from baseline to week 8 and EOT (end of treatment) for FMSD item scores:

TABLE 1
Change from baseline to week 8 and EOT (end of treatment) for FMSD items
(1) Difficulty with falling asleep, and (2) Restlessness of sleep

| | Week 8† | | EOT (LOCF) ‡ | |
|---|---|---|---|---|
| | | Compound of | | Compound of |
| | | Formula (I) | | Formula (I) |
| Analysis Set = FAS | Placebo | 15 mg | Placebo | 15 mg |

Difficulty with falling asleep

| | | | | |
|---|---|---|---|---|
| N | 81 | 76 | 94 | 90 |
| LS Mean (SE) | −0.90 (0.19) | −1.46 (0.19) | −0.81 (0.17) | −1.41 (0.18) |
| Treatment Difference (SE) | | −0.56 (0.26) | | −0.61 (0.25) |
| (Compound of Formula (I) - placebo) | | | | |
| 2-sided 90% CI for Difference | | (−0.99, −0.12) | | (−1.02, −0.20) |
| 1-sided p-value for Difference | | 0.018 | | 0.008 |

Restlessness of sleep

| | | | | |
|---|---|---|---|---|
| n | 81 | 76 | 94 | 90 |
| LS Mean (SE) | −1.05 (0.18) | −1.53 (0.19) | −0.94 (0.16) | −1.50 (0.17) |
| Treatment Difference (SE) | | −0.48 (0.26) | | −0.56 (0.23) |
| (Compound of Formula (I) - placebo) | | | | |
| 2-sided 90% CI for Difference | | (−0.90, −0.05) | | (−0.95, −0.17) |
| 1-sided p-value for Difference | | 0.033 | | 0.009 |

†Based on MMRM Analysis
‡ Based on ANCOVA analysis
Item scores range from 0 to 10. A negative change indicates an improvement from baseline in difficulty with falling asleep and restlessness of sleep.

TABLE 2
Change from baseline to week 8 and EOT (end of treatment) for FMSD items
(3) Difficulty getting comfortable, and (4) Difficulty staying asleep

| | Week 8† | | EOT (LOCF) ‡ | |
|---|---|---|---|---|
| | | Compound of | | Compound of |
| | | Formula (I) | | Formula (I) |
| Analysis Set = FAS | Placebo | 15 mg | Placebo | 15 mg |

Difficulty getting comfortable

| | | | | |
|---|---|---|---|---|
| n | 81 | 76 | 94 | 90 |
| LS Mean (SE) | −1.02 (0.16) | −1.24 (0.17) | −0.92 (0.15) | −1.27 (0.16) |
| Treatment Difference (SE) | | −0.23 (0.23) | | −0.35 (0.22) |
| (Compound of Formula (I) - placebo) | | | | |
| 2-sided 90% CI for Difference | | (−0.61, 0.16) | | (−0.71, 0.01) |
| 1-sided p-value for Difference | | 0.167 | | 0.054 |

Difficulty staying asleep

| | | | | |
|---|---|---|---|---|
| n | 81 | 76 | 94 | 90 |
| LS Mean (SE) | −0.95 (0.17) | −1.09 (0.17) | −0.86 (0.16) | −1.08 (0.16) |
| Treatment Difference (SE) | | −0.14 (0.24) | | −0.22 (0.22) |
| (Compound of Formula (I) - placebo) | | | | |
| 2-sided 90% CI for Difference | | (−0.53, 0.25) | | (−0.59, 0.14) |
| 1-sided p-value for Difference | | 0.275 | | 0.158 |

†Based on MMRM Analysis
‡ Based on ANCOVA analysis
Item scores range from 0 to 10. A negative change indicates an improvement from baseline for difficulty getting comfortable and difficulty staying asleep.

TABLE 3
Change from baseline to week 8 and EOT (end of treatment) for FMSD items (5) Degree
of deep sleep, and (6) Degree of being rested when waking up for the day

| | Week 8† | | EOT (LOCF) ‡ | |
|---|---|---|---|---|
| | | Compound of | | Compound of |
| | | Formula (I) | | Formula (I) |
| Analysis Set = FAS | Placebo | 15 mg | Placebo | 15 mg |

Degree of deep sleep

| | | | | |
|---|---|---|---|---|
| n | 81 | 76 | 94 | 90 |
| LS Mean (SE) | 0.45 (0.17) | 0.55 (0.17) | 0.45 (0.16) | 0.60 (0.16) |
| Treatment Difference (SE) | | 0.11 (0.24) | | 0.16 (0.22) |
| (Compound of Formula (I) - placebo) | | | | |
| 2-sided 90% CI for Difference | | (−0.29, 0.50) | | (−0.21, 0.53) |
| 1-sided p-value for Difference | | 0.331 | | 0.240 |

Degree of being rested when waking up for the day

| | | | | |
|---|---|---|---|---|
| n | 81 | 76 | 94 | 90 |
| LS Mean (SE) | 0.69 (0.18) | 0.74 (0.18) | 0.68 (0.16) | 0.78 (0.17) |
| Treatment Difference (SE) | | 0.04 (0.25) | | 0.10 (0.24) |
| (Compound of Formula (I) - placebo) | | | | |
| 2-sided 90% CI for Difference | | (−0.37, 0.46) | | (−0.29, 0.49) |
| 1-sided p-value for Difference | | 0.432 | | 0.337 |

†Based on MMRM Analysis
‡ Based on ANCOVA analysis
Item scores range from 0 to 10. A positive change indicates an improvement from baseline for degree of deep sleep and degree of being rested when waking up for the day.

TABLE 4
Change from baseline to week 8 and EOT (end of treatment) for FMSD items (7) Difficulty
with beginning the day, and (8) Degree of having enough sleep during the previous night

| | Week 8† | | EOT (LOCF) ‡ | |
|---|---|---|---|---|
| | | Compound of | | Compound of |
| | | Formula (I) | | Formula (I) |
| Analysis Set = FAS | Placebo | 15 mg | Placebo | 15 mg |

Difficulty with beginning the day

| | | | | |
|---|---|---|---|---|
| N | 81 | 76 | 94 | 90 |
| LS Mean (SE) | −0.50 (0.18) | −0.89 (0.19) | −0.49 (0.17) | −0.91 (0.18) |
| Treatment Difference (SE) | | −0.39 (0.26) | | −0.42 (0.25) |
| (Compound of Formula (I) - placebo) | | | | |
| 2-sided 90% CI for Difference | | (−0.82, 0.04) | | (−0.83, −0.01) |
| 1-sided p-value for Difference | | 0.070 | | 0.044 |

Degree of having enough sleep during the previous night

| | | | | |
|---|---|---|---|---|
| N | 81 | 76 | 94 | 90 |
| LS Mean (SE) | 0.80 (0.19) | 1.10 (0.19) | 0.77 (0.18) | 1.09 (0.18) |
| Treatment Difference (SE) | | 0.30 (0.27) | | 0.32 (0.25) |
| (Compound of Formula (I) - placebo) | | | | |
| 2-sided 90% CI for Difference | | (−0.14, 0.75) | | (−0.09, 0.73) |
| 1-sided p-value for Difference | | 0.131 | | 0.100 |

†Based on MMRM Analysis
‡ Based on ANCOVA analysis
Item scores range from 0 to 10. A negative change indicates an improvement from baseline for difficulty with beginning the day. A positive change indicates an improvement from baseline for degree of having enough sleep during the previous night.

### INCORPORATION BY REFERENCE

The entire disclosure of each of the patent documents and scientific articles referred to herein is incorporated by reference for all purposes.

### EQUIVALENTS

The disclosure can be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

We claim:

1. A method of treating a sleep disorder associated with pain in a patient in need thereof, the method comprising administering to the patient a compound of Formula (I), 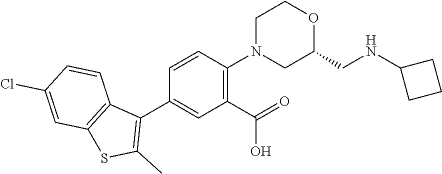 [SMILES: Cc1sc2cc(Cl)ccc2c1-c1ccc(N2CCO[C@H](CNC3CCC3)C2)c(C(=O)O)c1]
or a pharmaceutically acceptable salt thereof.

2. A method of treating a sleep disorder associated with pain in a patient in need thereof, the method comprising:
a) identifying and/or selecting a patient having a sleep disorder associated with pain; and
b) administering to the patient identified in step a) a compound of Formula (I), or a pharmaceutically acceptable salt thereof.

3. The method of any one of claims 1 to 2, wherein the compound of Formula I is in the form of the hydrobromide salt,

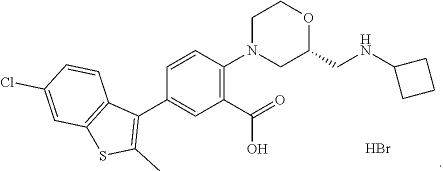 [SMILES: Cc1sc2cc(Cl)ccc2c1-c1ccc(N2CCO[C@H](CNC3CCC3)C2)c(C(=O)O)c1 ; Br]

4. The method of claim 3 wherein the method comprises administering the compound of Formula I, or a pharmaceutically acceptable salt thereof to a patient daily.

5. The method of any one of the preceding claims, wherein the compound of Formula (I) is administered in a therapeutically effective amount to improve sleep.

6. The method of any one of the preceding claims, wherein the compound of Formula (I) is administered in an amount equal to about 5 mg to about 45 mg per day, such as about 5 mg per day, about 10 mg per day, about 15 mg per day, about 20 mg per day, about 25 mg per day, about 30 mg per day, about 35 mg per day, about 40 mg per day, or about 45 mg per day.

7. The method of any one of the preceding claims, wherein the compound of Formula (I) is administered in an amount that is effective to treat the sleep disorder associated with pain, wherein the amount is not effective to treat the pain.

8. The method of any one of the preceding claims, wherein the sleep disorder is measured using a sleep diary, wrist actigraphy, or polysomnography.

9. The method of claim 8, wherein the sleep disorder is measured using a sleep diary, and the sleep disorder is selected from the group consisting of difficulty with falling asleep; restlessness of sleep; difficulty getting comfortable; difficulty staying asleep; degree of deep sleep; degree of being rested when waking up for the day; difficulty with beginning the day; degree of having enough sleep during the previous night.

10. The method of claim 8, wherein the sleep disorder is measured using wrist actigraphy or polysomnography, and the sleep disorder selected from the group consisting of difficulty with falling asleep; restlessness of sleep; difficulty staying asleep; degree of deep sleep.

11. The method of claim 8, wherein the sleep disorder is difficulty with falling asleep.

12. The method of claim 8, wherein the sleep disorder is restlessness of sleep.

13. The method of any one of the preceding claims, wherein the pain is selected from the group consisting of fibromyalgia, neuropathic pain, osteoarthritis, and visceral pain.

14. The method of claim 14, wherein the visceral pain is selected from the group consisting of IBS associated pain and bladder pain.

15. The method of any one of the preceding claims, wherein the patient is being treated with one or more of (a) a pain reliever selected from the group consisting of gabapentinoids (including pregabalin, gabapentin), antidepressants (except for serotonin reuptake inhibitors and especially duloxetine, venlafaxine, milnacipran, tricyclic antidepressants [especially amitriptyline and nortriptyline], trazodone, nefazodone, mirtazapine, bupropion), ketamine, esketamine and other NMDA receptor blocking drugs, GABAB receptor agonists (including sodium oxybate, baclofen), opioids (including morphine, fentanyl, codeine, hydrocodone, oxycodone, hydromorphone, and tramadol, celecoxib and meloxicam, muscle relaxants like cyclobenzaprine, cannabis and cannabinoids (especially those containing THC), NSAIDS (including acetaminophen or acetaminophen-containing formulations, ibuprofen, naproxen) chronic non-narcotic analgesics (with the exception of low dose aspirin for cardioprophylaxis, up to 325 mg daily) and topical pain medications (including capsaicin) (b) a procedure for pain relief selected from the group consisting of electrical stimulation including spinal cord stimulation or transcutaneous electrical nerve stimulation), acupuncture, nerve block, iontophoresis, laser therapy, tender point injections, dry needle injections chiropractic treatment, exercise or physical therapy, surgical therapy and biofeedback.

16. The method of any one of the preceding claims, wherein the method reduces the severity of a symptom of the sleep disorder.

17. The method of any one of the preceding claims, wherein the method reduces the severity of a symptom of the sleep disorder by at least 15%, at least 20%, at least 25%, at least 30%, at least 35% or at least 40%.

18. The method of any one of claims 1 to 17, wherein the method comprises determining the value for the patient of a clinical score associated with the sleep disorder.

19. The method of claim 18, wherein the clinical score is a score for one or more FMSD items.

20. The method of claim 19, wherein the method comprises determining that the value of an FMSD item at a time point following administration of the compound of Formula (I) is different from the value of the FMSD item prior to or at the time of administration.

21. The method of claim 19, wherein the method comprises determining that the value of an FMSD item at least 1 week, such as from 1 week to 13 weeks, following administration of the compound of Formula (I) is different from the value of the FMSD item prior to or at the time of administration.

22. The method of claim 20 or 21, wherein the value of the FMSD item following administration of the compound of Formula (I) differs by at least 15%, at least 20%, at least 25%, at least 30%, at least 35% or at least 40%. from the value of the FMSD item prior to or at the time of administration.

23. The method of claim 22, wherein the value of an FMSD item at least 1 week, such as from 1 week to 13 weeks, following administration of the compound of Formula (I) increases by at least 15%, at least 20%, at least 25%, at least 30%, at least 35% or at least 40% relative to the value of the FMSD item prior to or at the time of administration, wherein the FMSD item is selected from the group consisting of degree of deep sleep, degree of being rested when waking up for the day, and degree of having enough sleep during the previous night.

24. The method of claim 22, wherein the value of an FMSD item at least 1 week, such as from 1 week to 13 weeks, following administration of the compound of Formula (I) decreases by at least 15%, at least 20%, at least 25%, at least 30%, at least 35% or at least 40% relative to the value of the FMSD item prior to or at the time of administration, wherein the FMSD item is selected from the group consisting of difficulty with falling asleep, restlessness of sleep, difficulty getting comfortable, difficulty staying asleep, and difficulty with beginning the day.

25. The method of claim 19, wherein the method comprises determining that, for each of at least two FMSD items, the value of the FMSD item at a time point following administration of the compound of Formula (I) is different from the value of the FMSD item prior to or at the time of administration.

26. The method of claim 19, wherein the method comprises determining that, for each of at least two FMSD items, the value of the FMSD item at least 1 week, such as from 1 week to 13 weeks, following administration of the compound of Formula (I) is different from the value of the FMSD item prior to or at the time of administration.

27. The method of claim 25 or 26, wherein for each of at least two FMSD items, the value of the FMSD item following administration of the compound of Formula (I) differs by at least 15%, at least 20%, at least 25%, at least 30%, at least 35% or at least 40% from the value of the FMSD item prior to or at the time of administration.

28. The method of claim 27, wherein the method comprises determining that for each of at least two FMSD items, the value of the FMSD item at least 1 week, such as from 1 week to 13 weeks, following administration of the compound of Formula (I) increases by at least 15%, at least 20%, at least 25%, at least 30%, at least 35% or at least 40% relative to the value of the FMSD item prior to or at the time of administration, wherein the FMSD item is selected from the group consisting of degree of deep sleep, degree of being rested when waking up for the day, and degree of having enough sleep during the previous night.

29. The method of claim 27, wherein the method comprises determining that for each of at least two FMSD items, the value of the FMSD item at least 1 week, such as from 1 week to 13 weeks, following administration of the compound of Formula (I) decreases by at least 15%, at least 20%, at least 25%, at least 30%, at least 35% or at least 40% relative to the value of the FMSD item prior to or at the time of administration, wherein the FMSD item is selected from the group consisting of difficulty with falling asleep, restlessness of sleep, difficulty getting comfortable, difficulty staying asleep, and difficulty with beginning the day.
